SEQUENCE LISTING (10 sequences)

SEQ ID NO: 1 - DNA, 1422 bp, Artificial Sequence
  Thermoanaerobacter sp. 16SrDNA
ggttgggtcaccggcttcgggtgtcgcaggctctcgtggtgtgacgggcggtgtgtacaaggcccgggaacgtattcaccgcggcatgctgatccgcgattactagcgattccgacttcatgcaggcgagttgcagcctgcaatccgaacttggaccggctttttgggattcgctccgcctcacggcttcgcttccctctgtaccggccattgtagcacgtgtgtggcccagggcatttagggcatgatgatttgacgtcatccccaccttcctccgtgtcctccacggcagtccctctagagtgcccggcttacccgctggcaactagaggcaggggttgcgctcgttgcgggacttaacccaacatctcacgacacgagctgacgacaaccatgcaccacctgtgcaggctccttacctcccggtaaggtcgctcccctttcggttcgctactacctgcatgtcaagccctggtaaggttcttcgcgttgcttcgaattaaaccacatgctccaccgcttgtgcgggcccccgtcaattcctttgagtttcaaccttgcggccgtactccccaggcggggtacttattgcgttcgctacggcacggaacgcttccgcgccccacacctagtacccatcgtttacagcgtggactaccagggtatctaatcctgttcgctccccacgctttcgcgcctcagcgtcagggccagtccagagagtcgccttcgccactggtattcctcccgatatctacgcatttcaccgctacaccgggaattccactcccctctcctgccctctagccaatcagtttcagatgctaccccccggttgagcccgggtcttttacacctgacttgattgaccgcctacgcgccctttacgcccagtaattccggacaacgctcgccccctacgtcttaccgcggctgctggcacgtagttagccggggctttcgtgtggtaccgtcatcccttcttcccacactaacggggtttacaacccgaaggccttcctcccccacgcggcgtcgctgggtcaggcttccgcccattgcccaagattccccactgctgcctcccgtaggagtctgggccgtgtctcagtcccagtgtggccgtccaccctctcaggccggctacccgtcgtcgccttggtaggccgttaccctaccaactagctgatgggacgcgggcccatccttaagcggtagcttgcgcttccctttcctccctataggatgccctataaggagcttatccagtattaccacccctttcgaggtgctatcccggtcttaagggtaggttgcccacgcgttactcacccgtccgccgctatccgccacccaactacgttgagtgccggaccgctcgactgcatgtgttaggcacgccgccagcgttcgtcctgagcc

SEQ ID NO: 2 - DNA, 1659 bp, Artificial Sequence
  Thermoanaerobacter sp. 16SrDNA
actcaagtgggcacgtttttttctcttcatcacgtttctaacatgcccacttgagtgccgggttgggtcaccggcttcgggtgttgcagactctcgtggtgtgacgggcggtgtgtacaaggcccgggaacgtattcaccgcggcatgctgatccgcgattactagcgattccgacttcatgcaggcgagttgcagcctgcaatccgaacttggaccggctttttggggtccgctccagatcgctccttcgcctccctctgtaccggccattgtagcacgtgtgtggcccagggcatatagggcatgatgatttgacgtcatccccaccttcctccgtgttgtccacggcagtccctctagagtgcctccgtcactcaactgaacacgctatcccttcctctctactctttcctaacatgttcagttgagtgacggactggcaactagaagcaagggttgcgctcgttgcgggacttaacccaacatctcacgacacgagctgacgacaaccatgcaccacctgtgcaggctcccggcactcaagtaggcacttcattctccctcttactaccttctctatcatgcccacttgagtgccgggtcgctcacctttcggctcgctactacctgcatgtcaagccctggtaaggttcttcgcgttgcttcgaattaaaccacatgctccaccgcttgtgcgggcccccgtcaattcctttgagtttcaaccttgcggccgtactccccaggcggggtacttattgcgttaactacggcacggaatgcttccgcatcccacacctagtacccatcgtttacggcgtggactaccagggtatctaatcctgtttgctccccacgctttcgcgcctcagcgtcagggtcagtccagagagtcgccttcgccactggtattcctcccgatatctacgcatttcaccgctacaccgggaattccactcccctctcctgccctctagccacccagtttcatgtgcatcccccgggttgagcccgggttttttacacctgacttaagtggccgcctacgcgccctttacgcccagtaattccggacaacgctcgccccctacgtcttaccgcggctgctggcacgtagttagccggggctttcgtgtggtaccgtcatctattcttcccacactatcgagctttacgacccgaaggccttcttcgctcacgcggcgtcgctgcgtcaggctttcgcccattgcgcaagattccccactgctgcctcccgtaggagtctgggccgtgtctcagtcccagtgtggccgaccaccctctcaggccggctacccgtcgtcgccttggtaggccgttaccctaccaactagctgatgggacgcgggcccatccttaagcggtagcttccgctaccttccctcctcataggatgccctacaaggagcttatccagtattagcacccctttcgaggtgttatcccggtcttaagggtaggttgcccacgcgttactcacccgtccgccgctatccggcactcaactccgtgcttaccttactttgcaccacttttattactttcttcttctactatacttccttccccttaagtaagcacttagttgagtgccggaccgctcgacttgcatgtgttaggcacgccgccagcgttcg

SEQ ID NO: 3 - DNA, 1436 bp, Artificial Sequence
  Thermoanaerobacter sp. 16SrDNA
cccggttgggtcaccggcttcgggtgtcgcaggctctcgtggtgtgacgggcggtgtgtacaaggcccgggaacgtattcaccgcggcatgctgatccgcgattactagcgattccgacttcatgcaggcgagttgcagcctgcaatccgaacttggaccggctttttgggattcgctccgcctcgcggcttcgctcccctctgtaccggccattgtagcacgtgtgtggcccagggcatatagggcatgatgatttgacgtcatccccaccttcctccgtgtcctccacggcagtccccctagagtgcccggcttacccgctggcaactagaggcaggggttgcgctcgttgcgggacttaacccaacatctcacgacacgagctgacgacaaccatgcaccacctgtgcaggctccttacctcccggtaaggtcgctcccctttcggttcgctactacctgcatgtcaagccctggtaaggttcttcgcgttgcttcgaattaaaccacatgctccaccgcttgtgcgggcccccgtcaattcctttgagtttcaaccttgcggccgtactccccaggcggggtacttattgcgttcgctacggcacggaacgcttccgcgccccacacctagtacccatcgtttacagcgtggactaccagggtatctaatcctgttcgctccccacgctttcgcgcctcagcgtcagggccagtccagagagtcgccttcgccactggtattcctcccgatatctacgcatttcaccgctacaccgggaattccactcccctctcctgccctctagccaatcagtttcagatgctacccccgggttgagcccgggtcttttacacctgacttgattgaccgcctacgcgccctttacgcccagtaattccggacaacgctcgccccctacgtcttaccgcggctgctggcacgtagttagccggggctttcgtgtggtaccgtcatcccttcttcccacactaacggggtttacaacccgaaggccttcctcccccacgcggcgtcgctgggtcaggcttccgcccattgcccaagattccccactgctgcctcccgtaggagtctgggccgtgtctcagtcccagtgtggccgaccaccctctcaggccggctacccgtcgtcgccttggtaggccgttaccctaccaactagctgatgggacgcgggcccatccttaagcggtagcttgcgcctccctttcctccctataggatgccctataaggagcttatccagtattaccacccctttcgaggtgctatcccggtcttaagggtaggttgcccacgcgttactcacccgtccgccgctatccgccacccaactacgttgagtgccggaccgctcgacttgcatgtgttaggcacgccgccagcgttcgtcctgagccatgatcaaac

SEQ ID NO: 4 - DNA, 1080 bp, Artificial Sequence
  Thermoanaerobacter sp. 16SrDNA
gctcaggacgaacgctggcggcgtgcctaacacatgcaagtcgagcggtccggcactcaactaagtgcttacttaaggggaaggaagtatagtagaagaagaaggtaataaaagtgatgcaaagtaaggtaagcacggagttgagtgccggatagcggcggacgggtgagtaacgcgtgggcaacctacccttaagaccgggataacacctcgaaaggggtgctaatactggataagctccttgtagggcatcctatgaggagggaaggtagcggaagctaccgcttaaggatgggcccgcgtcccatcagctagttggtagggtaacggcctaccaaggcgacgacgggtagccggcctgagagggtggtcggccacactgggactgagacacggcccagactcctacgggaggcagcagtggggaatcttgcgcaatgggcgaaagcctgacgcagcgacgccgcgtgagcgaagaaggccttcgggtcgtaaagctcgatagtgtgggaagaatagatgacggtaccacacgaaagccccggctaactacgtgccagcagccgcggtaagacgtagggggcgagcgttgtccggaattactgggcgtaaagggcgcgtaggcggccacttaagtcaggtgtaaaaaacccgggctcaacccgggggatgcacatgaaactgggtggctagagggcaggagaggggagtggaattcccggtgtagcggtgaaatgcgtagatatcgggaggaataccagtggcgaaggcgactctctggactgaccctgacgctgaggcgcgaaagcgtggggagcaaacaggattagataccctggtagtccacgccgtaaacgatgggtactaggtgtgggatgcggaagcattccgtgccgtagttaacgcaataagtaccccgcctggggagtacggccgcaaggttgaaactcaaaggaattgacgggggcccgcacaagcggtggagcatgtggtttaattcgaagcaacgcgaagaaccttaccagggcttgacatgcaggtagtagcgagccgaaaggtgagcgacccggcactcaagtg

SEQ ID NO: 5 - DNA, 1451 bp, Artificial Sequence
  Thermoanaerobacter sp. 16SrDAN
gccccactttcgacggctccctccttcccggttgggtcaccggcttcgggtgtcgcaggctctcgtggtgtgacgggcggtgtgtacaaggcccgggaacgtattcaccgcggcatgctgatccgcgattactagcgattccgacttcatgcaggcgagttgcagcctgcaatccgaacttggaccggctttttgggattcgctccgcctcgcggcttcgcttccctctgtaccggccattgtagcacgtgtgtggcccagggcatatagggcatgatgatttgacgtcatccccaccttcctccgtgtcctccacggcagtccctctagagtgcccggcttacccgctggcaactagaggcaggggttgcgctcgttgcgggacttaacccaacatctcacgacacgagctgacgacaaccatgcaccacctgtgcaggctccttacctcccggtaaggtcgctcccctttcggttcgctactacctgcatgtcaagccctggtaaggttcttcgcgttgcttcgaattaaaccacatgctccaccgcttgtgcgggcccccgtcaattcctttgagtttcaaccttgcggccgtactccccaggcggggtacttattgcgttcgctacggcacggaacgcttccgcgccccacacctagtacccatcgtttacagcgtggactaccagggtatctaatcctgttcgctccccacgctttcgcgcctcagcgtcagggccagtccagagagtcgccttcgccactggtattcctcccgatatctacgcatttcaccgctacaccgggaattccactcccctctcctgccctctagccaatcagtttcagatgctacccccgggttgagcccgggtcttttacacctgacttgattgaccgcctacgcgccctttacgcccagtaattccggacaacgctcgccccctacgtcttaccgcggctgctggcacgtagttagccggggctttcgtgtggtaccgtcatcccttcttcccacactaacggggtttacaacccgaaggccttcctcccccacgcggcgtcgctgggtcaggcttccgcccattgcccaagattccccactgctgcctcccgtaggagtctgggccgtgtctcagtcccagtgtggccgaccaccctctcaggccggctacccgtcgtcgccttggtaggccgttaccctaccaactagctgatgggacgcgggcccatccttaagcggtagcttgcgcctccctttcctccctataggatgccctataaggagcttatccagtattaccacccctttcgaggtgctatcccggtcttaagggtaggttgcccacgcgttactcacccgtccgccgctatccgccacccaactacgttgagtgccggaccgctcgacttgcatgtgttaggcacgccgccagcgttcgtcctgagc

SEQ ID NO: 6 - DNA, 1465 bp, Artificial Sequence
  Thernoanaerobacter sp. 16SrDNA
ttcaccccaatcacctgccccaccttcgacggctccctcctccccggttgggtcaccggcttcgggtgtcgcaggctctcgtggtgtgacgggcggtgtgtacaaggcccgggaacgtattcaccgcggcatgctgatccgcgattactagcgattccgacttcatgcaggcgagttgcagcctgcaatccgaacttggaccggctttttgggattcgctccgcctcgcggcttcgctcccctctgtaccggccattgtagcacgtgtgtggcccagggcatatagggcatgatgatttgacgtcatccccaccttcctccgtgtcctccacggcagtccccctagagtgcccggcttacccgctggcaactagaggcaggggttgcgctcgttgcgggacttaacccaacatctcacgacacgagctgacgacaaccatgcaccacctgtgcaggctccttacctcccggtaaggtcgctcccctttcggttcgctactacctgcatgtcaagccctggtaaggttcttcgcgttgcttcgaattaaaccacatgctccaccgcttgtgcgggcccccgtcaattcctttgagtttcaaccttgcggccgtactccccaggcggggtacttattgcgttcgctacggcacggaacgcttccgcgccccacacctagtacccatcgtttacagcgtggactaccagggtatctaatcctgttcgctccccacgctttcgcgcctcagcgtcagggccagtccagagagtcgccttcgccactggtattcctcccgatatctacgcatttcaccgctacaccgggaattccactcccctctcctgccctctagccaatcagtttcagatgctacccccgggttgagcccgggtcttttacacctgacttgattgaccgcctacgcgccctttacgcccagtaattccggacaacgctcgccccctacgtcttaccgcggctgctggcacgtagttagccggggctttcgtgtggtaccgtcatcccttcttcccacactaacggggtttacaacccgaaggccttcctcccccacgcggcgtcgctgggtcaggcttccgcccattgcccaagattccccactgctgcctcccgtaggagtctgggccgtgtctcagtcccagtgtggccgaccaccctctcaggccggctacccgtcgtcgccttggtaggccgttaccctaccaactagctgatgggacgcgggcccatccttaagcggtagcttgcgcctccctttcctccctataggatgccctataaggagcttatccagtattaccacccctttcgaggtgctatcccggtcttaagggtaggttgcccacgcgttactcacccgtccgccgctatccgccacccaactacgttgagtgccggaccgctcgacttgcatgtgttaggcacgccgccagcgttcgtcctga

SEQ ID NO: 7 - DNA, 1665 bp, Artificial Sequence
  Thermoanaerobacter sp. 16SrDNA
actcaagtgggcacgtttttttctcttcatcacgtttctaacatgcccacttgagtgccgggttgggtcaccggcttcgggtgttgcagactctcgtggtgtgacgggcggtgtgtacaaggcccgggaacgtattcaccgcggcatgctgatccgcgattactagcgattccgacttcatgcaggcgagttgcagcctgcaatccgaacttggaccggctttttggggtccgctccagatcgctccttcgcctccctctgtaccggccattgtagcacgtgtgtggcccagggcatatagggcatgatgatttgacgtcatccccaccttcctccgtgttgtccacggcagtccctctagagtgcctccgtcactcaactgaacacgctatcccttcctctctactctttcctaacatgttcagttgagtgacggactggcaactagaagcaagggttgcgctcgttgcgggacttaacccaacatctcacgacacgagctgacgacaaccatgcaccacctgtgcaggctcccggcactcaagtaggcacttcattctccctcttactaccttctctatcatgcccacttgagtgccgggtcgctcacctttcggctcgctactacctgcatgtcaagccctggtaaggttcttcgcgttgcttcgaattaaaccacatgctccaccgcttgtgcgggcccccgtcaattcctttgagtttcaaccttgcggccgtactccccaggcggggtacttattgcgttaactacggcacggaatgcttccgcatcccacacctagtacccatcgtttacggcgtggactaccagggtatctaatcctgtttgctccccacgctttcgcgcctcagcgtcagggtcagtccagagagtcgccttcgccactggtattcctcccgatatctacgcatttcaccgctacaccgggaattccactcccctctcctgccctctagccacccagtttcatgtgcatcccccgggttgagcccgggttttttacacctgacttaagtggccgcctacgcgccctttacgcccagtaattccggacaacgctcgccccctacgtcttaccgcggctgctggcacgtagttagccggggctttcgtgtggtaccgtcatctattcttcccacactatcgagctttacgacccgaaggccttcttcgctcacgcggcgtcgctgcgtcaggctttcgcccattgcgcaagattccccactgctgcctcccgtaggagtctgggccgtgtctcagtcccagtgtggccgaccaccctctcaggccggctacccgtcgtcgccttggtaggccgttaccctaccaactagctgatgggacgcgggcccatccttaagcggtagcttccgctaccttccctcctcataggatgccctacaaggagcttatccagtattagcacccctttcgaggtgttatcccggtcttaagggtaggttgcccacgcgttactcacccgtccgccgctatccggcactcaactccgtgcttaccttactttgcaccacttttattactttcttcttctactatacttccttccccttaagtaagcacttagttgagtgccggaccgctcgacttgcatgtgttaggcacgccgccagcgttcgtcctga

SEQ ID NO: 8 - DNA, 1105 bp, Artificial Sequence
  Thermoanaerobacter sp. 16SrDNA
tcaggacgaacgctggcggcgtgcctaacacatgcaagtcgagcggtccggcactcaacgtagttgagtggcggatagcggcggacgggtgagtaacgcgtgggcaacctacccttaagaccgggatagcacctcgaaaggggtggtaatactggataagctccttatagggcatcctatagggaggaaagggaagcgcaagctaccgcttaaggatgggcccgcgtcccatcagctagttggtagggtaacggcctaccaaggckacgacgggtagccggcctgagagggtggtcggccacactgggactgagacacggcccagactcctacgggaggcagcagtggggaatcttgggcaatgggcggaagcctgacccagcgacgccgcgtgggggaggaaggccttcgggttgtaaaccccgttagtgtgggaagaagggatgacggtaccacacgaaagccccggctaactacgtgccagcagccgcggtaagacgtagggggcgagcgttgtccggaattactgggcgtaaagggcgcgtaggcggtcaatcaagtcaggtgtaaaagacccgggctcaacccgggggtagcacctgaaactggttggctagagggcaggagaggggagtggaattcccggtgtagcggtgaaatgcgtagatatcgggaggaataccagtggcgaaggcgactctctggactggccctgacgctgaggcgcgaaagcgtggggagcgaacaggattagataccctggtagtccacgctgtaaacgatgggtactaggtgtggggcgcggaagcgttccgtgccgtagcgaacgcaataagtaccccgcctggggagtacggccgcaaggttgaaactcaaaggaattgacgggggcccgcacaagcggtggagcatgtggtttaattcgaagcaacgcgaagaaccttaccagggcttgacatgcaggtggtagcgaaccgaaaggtgagcgaccttaccgggaggtaaggagcctgcacaggtggtgcatggttgtcgtcagctcgtgtcgtgagatgttgggttaagtcccgcaacgagcgcaacccctgcctctagttgccagcgg

SEQ ID NO: 9 - DNA, 20 bp, Artificial Sequence
  27F forward primer
agagtttgatcmtggctcag

SEQ ID NO: 10 - DNA, 19 bp, Artificial Sequence
  1492R reverse primer
ggttaccttgttacgactt

(19) United States
(12) Patent Application Publication
(10) Pub. No.: US 2014/0377821 A1
(43) Pub. Date: Dec. 25, 2014

(54) BIOCONVERSION OF LIGNOCELLULOSIC BIOMASS TO LACTIC ACID

(71) Applicant: DIREVO Industrial Biotechnology GmbH, Köln (DE)

(72) Inventors: Vitaly Svetlichnyi, Koln (DE); Simon Curvers, Koln (DE)

(21) Appl. No.: 14/349,306

(22) PCT Filed: Oct. 7, 2012
(86) PCT No.: PCT/EP2012/069810

§ 371 Date: Apr. 2, 2014

Related U.S. Application Data
(60) Provisional application No. 61/544,831, filed on Oct. 7, 2011.
(60) Provisional application No. 61/556,448, filed on Nov. 7, 2011.
(60) Provisional application No. 61/669,998, filed on Jul. 10, 2012.

(30) Foreign Application Priority Data
Oct. 7, 2011 (EP) 11008155.1
Nov. 7, 2011 (EP) 11008857.2
Jul. 10, 2012 (EP) 12175684.5

(51) Int. Cl. C12P 7/56

(52) U.S. Cl. CPC C12P 7/56; C12P 2201/00; C12P 2203/00
USPC 435/139

(57) ABSTRACT

The present technology pertains to methods for processing lignocellulosic hydrolysates to valuable carboxylic acids using novel xylanolytic, amylolytic and saccharolytic thermophilic bacterial strains belonging to the genus Thermoanaerobacter.

### FIELD OF THE DISCLOSURE

The present disclosure pertains to methods for producing valuable carboxylic acids like lactic acid, salts or esters thereof from poly-, oligo-, di-, and/or monosaccharides derived from lignocellulosic biomass using novel xylanolytic, amylolytic and saccharolytic thermophilic bacterial strains belonging to the genus Thermoanaerobacter.

### BACKGROUND

Many carboxylic acids are, produced industrially on a large scale. They are also pervasive in nature. Carboxylic acids are used in the production of polymers, pharmaceuticals, solvents, and food additives. Industrially important carboxylic acids include acetic acid (component of vinegar, precursor to solvents and coatings), acrylic and methacrylic acids (precursors to polymers, adhesives), adipic acid (polymers), citric acid (beverages), ethylenediaminetetraacetic acid (chelating agent), fatty acids (coatings), maleic acid (polymers), propionic acid (food preservative), terephthalic acid (polymers).

Lactic acid is widely used in food, pharmaceutical and textile industries. It is also used as a source of lactic acid polymers which are being used as biodegradable plastics. The physical properties and stability of polylactides can be controlled by adjusting the proportions of the L(+)- and D(−)-lactides. Optically pure lactic acid is currently produced by the fermentation of glucose derived from cornstarch using various lactic acid bacteria 7.

However, the fastidious lactic acid bacteria have complex nutritional requirements and the use of corn as the feedstock competes directly with the food and feed uses. Lignocellulosic biomass represents a potentially inexpensive and renewable source of sugars for fermentation. Therefore, the industry of producing fermentation products such as lactic acid is facing the challenge of redirecting the production process from fermentation of relatively easily convertible but expensive starchy materials, to the complex but inexpensive lignocellulosic biomass such as plant biomass.

Unlike starch, which contains homogenous and easily hydrolyzed polymers, lignocellulosic biomass contains variable amounts of cellulose, hemicellulose, lignin and small amounts of protein, pectin, wax and other organic compounds. Lignocellulosic biomass should be understood in its broadest sense, so that it apart from wood, agricultural residues, energy crops also comprises different types of waste from both industry and households. Cellulosic biomass is a vast poorly exploited resource, and in some cases a waste problem. However, hexoses from cellulose can be converted by yeast to lactic acid for which there is a growing demand. Pentoses from hemicellulose cannot yet be converted to lactate commercially but several promising microorganisms with the capacity to convert pentoses and hexoses are under development.

Typically, the first step in utilization of lignocellulosic biomass is a pretreatment step, in order to fractionate the components of lignocellulosic material and increase their surface area. The pretreatment method most often used is steam pretreatment, a process comprising heating of the lignocellulosic material by steam injection to a temperature of 130-230° C. Prior to or during steam pretreatment, a catalyst like mineral or organic acid or a caustic agent facilitating disintegration of the biomass structure can be added optionally.

Another type of lignocellulose hydrolysis is acid hydrolysis, where the lignocellulosic material is subjected to an acid such as sulphuric acid whereby the sugar polymers cellulose and hemicellulose are partly or completely hydrolysed to their constituent sugar monomers and the structure of the biomass is destroyed facilitating access of hydrolytic enzymes in subsequent processing steps.

A further method is wet oxidation wherein the material is treated with oxygen at 150-185° C. Either pretreatment can be followed by enzymatic hydrolysis to complete the release of sugar monomers. This pre-treatment step results in the hydrolysis of cellulose into glucose while hemicellulose is transformed into the pentoses xylose and arabinose and the hexoses glucose, mannose and galactose. Thus, in contrast to starch, the hydrolysis of lignocellulosic biomass results in the release of pentose sugars in addition to hexose, sugars. This implies that useful fermenting organisms need to be able to convert both hexose and pentose sugars to desired fermentation products such as lactate.

After the pre-treatment the lignocellulosic biomass processing schemes involving enzymatic or microbial hydrolysis commonly involve four biologically mediated transformations: (1) the production of saccharolytic enzymes (cellulases and hemicellulases); (2) the hydrolysis of carbohydrate components present in pretreated biomass to sugars; (3) the fermentation of hexose sugars (e.g. glucose, mannose, and galactose); and (4) the fermentation of pentose sugars (e.g., xylose and arabinose).

Each processing step can make the overall process more costly and, therefore, decrease the economic feasibility of producing carbon-based chemicals from cellulosic biological material. Thus, there is a need to develop methods that reduce the number of processing steps needed to convert cellulosic biological material to biofuel and other commercially desirable materials.

The four biologically mediated transformations may occur in a single step in a process configuration called consolidated bioprocessing (CBP), which is distinguished from other less highly integrated configurations in that CBP does not involve a dedicated process step for cellulase and/or hemicellulase production. CBP offers the potential for higher efficiency than a processes requiring dedicated cellulase production in a distinct unit operation.

Therefore, the availability of novel microorganisms and methods for converting lignocellulosic biomass material to valuable carboxylic acids would be highly advantageous.

### SUMMARY OF THE DISCLOSURE

The present disclosure relates to methods for processing lignocellulosic hydrolysates to carboxylic acids like lactic acid, salts or esters thereof.

In a first aspect the present invention pertains to methods for converting hydrolyzed lignocellulosic biomass material to carboxylic acids comprising the step of contacting the hydrolyzed lignocellulosic biomass material with a microbial culture for a period of time at an initial temperature and an initial pH, thereby producing an amount of a carboxylic acids like lactic acid, wherein the microbial culture comprises an extremely thermophilic strain of the genus Thermoanaerobacter.

In one aspect, embodiments of this disclosure relate to the use of the extremely thermophilic strain strains Thermoanaerobacter sp. DIB004G, Thermoanaerobacter sp. DIB087G, Thermoanaerobacter sp. DIB097X, Thermoanaerobacter sp. DIB101G, Thermoanaerobacter sp. DIB101X, Thermoanaerobacter sp DIB103X, Thermoanaerobacter sp. DIB DIB104X and/or Thermoanaerobacter sp. DIB107X in a method according to the present disclosure, each respectively characterized by having a 16S rDNA sequence at least 99 to 100%, preferably 99.5 to 99.99 percent identical to SEQ ID NO. 1, SEQ ID NO. 2, SEQ ID NO. 3, SEQ ID NO. 4, SEQ ID NO. 5, SEQ ID NO. 6, SEQ ID NO. 7 or SEQ ID NO 8 as outlined in table 1.

Accordingly, the present disclosure pertains to the use of a Thermoanaerobacter sp. strain for processing lignocellulosic hydrolysates to carboxylic acids, preferably to lactic acid, selected from the group consisting of DIB004G, DIB087G, DIB097X, DIB101G, DIB101X, DIB103X, DIB104X and DIB107X, all listed with their respective accession numbers and deposition dates in table 1, and homologues microorganisms derived therefrom, progenies or mutants thereof.

The disclosure is based on the use of the isolated Thermoanaerobacter strains DIB097X, DIB101X, DIB103X, DIB104X and DIB107X, which are capable of growing and producing high levels of carbon based fermentation products from lignocellulosic hydrolysates and/or directly from poly-, oligo, di- and/or monosaccharides, in particular from poly-, oligo, di- and/or monosaccharides derived from pre-treated lignocellulosic biomass with polysaccharides being limited to hemicelluloses, e.g. xylan and starch.

The disclosure is further based on the use of the isolated Thermoanaerobacter sp. strains DIB004G, DIB087G and DIB101G which are capable of growing and producing high levels of carbon based fermentation products from lignocellulosic hydrolysates and/or directly from poly-, oligo, di- and/or monosaccharides, in particular from poly-, oligo, di- and/or monosaccharides derived from pre-treated lignocellulosic biomass with polysaccharides being limited to starch.

The used microorganisms according to the present disclosure and mutants thereof have broad substrate specificity, and are capable of utilizing pentoses such as xylose and arabinose and of hexoses such as glucose, mannose, fructose and galactose. The strains further have the advantage of being extremely thermophilic and thus are capable of growing at very high temperatures resulting in high productivities and substrate conversion rates, low risk of contamination and facilitated product recovery.

The used novel isolated microorganisms are saccharolytic and amylolytic or saccharolytic, amylolytic and xylanolytic, respectively, and belonging to the genus Thermoanaerobacter, in particular the microorganisms are capable of producing high levels of lactic acid from lignocellulosic hydrolysates while producing low levels of acetic acid.

In still another aspect, embodiments of this disclosure relate to methods for converting lignocellulosic hydrolysates to a carboxylic acid like lactic acid comprising the step of contacting the lignocellulosic hydrolysates with a microbial culture for a period of time at an initial temperature and an initial pH, thereby producing an amount of acarboxylic acid or other carbon-based products; wherein the microbial culture comprises an extremely thermophilic microorganism of the genus Thermoanaerobacter, in particular any microorganism of the strain Thermoanaerobacter sp. listed in table 1 with their respective accession numbers, microorganisms derived there from or homologous thereof.

In another aspect, embodiments of this disclosure relate to methods for converting starch or starch-containing feedstock to a carboxylic acids like lactic acid comprising the step of contacting the starch-containing feedstock with a microbial culture for a period of time at an initial temperature and an initial pH, thereby producing an amount of carboxylic acid like lactic acid; wherein the microbial culture comprises an extremely thermophilic microorganism of the genus Thermoanaerobacter, in particular any microorganism of the strain Thermoanaerobacter sp. listed in table 1 with their respective accession numbers, microorganisms derived there from or homologous thereof.

In still another aspect, embodiments of this disclosure relate to methods for converting a combination or mixture of lignocellulosic hydrolysates and starch-containing feedstock to a carboxylic acid, preferably lactic acid, a salt or an ester thereof, comprising the step of contacting the mixture with a microbial culture for a period of time at an initial temperature and an initial pH, thereby producing an amount of carboxylic acid; wherein the microbial culture comprises an extremely thermophilic microorganism of the genus Thermoanaerobacter, in particular any microorganism of the strain Thermoanaerobacter sp. listed in table 1 with their respective accession numbers, microorganisms derived there from or homologous thereof.

Before the disclosure is described in detail, it is to be understood that this disclosure is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. It is moreover to be understood that, in case parameter ranges are given which are delimited by numeric values, the ranges are deemed to include these limitation values.

To provide a comprehensive disclosure without unduly lengthening the specification, the applicant hereby incorporates by reference each of the patents and patent applications cited herein.

### BRIEF DESCRIPTION OF THE DRAWINGS

FIG. 1 illustrates a phylogenetic tree based on 16S rDNA genes for all Thermoanaerobacter sp. strains comprised in the invention as listed in table 1

FIG. 2 shows a table indicating performance data from all strains listed in table 1 during cultivation on cellobiose, glucose, xylane and xylose.

FIG. 3 shows a table indicating performance data from all strains listed in table 1 during cultivation on pretreated poplar wood and performance data from selected strains DIB004G and DIB097X on different lignocellulosic feedstock types.

FIG. 4 shows a graph displaying formation of lactate during growth of Thermoanaerobacter sp. DIB097X on pretreated miscanthus grass.

FIG. 5 shows a graph displaying formation of lactate during growth of Thermoanaerobacter sp. DIB004C on ground corn seed.

### DETAILED DESCRIPTION OF THIS DISCLOSURE

As mentioned above, the present disclosure relates to methods for processing lignocellulosic hydrolysates. The disclosure relates, in certain aspects, to the use of microorganisms, which are able to convert pretreated lignocellulosic biomass such as, for example, poplar wood chips or miscanthus grass, to an economically desirable product such as, for example, a carboxylic acid, in particular to lactic acid, a salt or an ester thereof like lactate.

Furthermore, the present disclosure relates to methods for converting sugars like poly-, oligo, di- and/or mono-saccharides, in particular poly-, oligo, di- and/or mono-saccharides of hexoses and/or poly-, oligo, di- and/or monosaccharides of pentoses to produce carbon based chemicals like carboxylic acid, in particular to lactic acid, a salt or an ester thereof like lactate.

Carboxylic acids are organic acids characterized by the presence of at least one carboxyl group. The general formula of a carboxylic acid is R—COOH, where R is some monovalent functional group. A carboxyl group (or carboxy) is a functional group consisting of a carbonyl (RR′C═O) and a hydroxyl (R—O—H), which has the formula —C(═O)OH, usually written as —COOH or —CO2H. The term carboxylic acids includes salts and esters of the acids. Lactic acid is a carboxylic acid with the chemical formula C₃H₆O₃. It has a hydroxyl group adjacent to the carboxyl group, making it an alpha hydroxy acid (AHA).

The present inventors have found microorganisms of the genus Thermoanaerobacter which have a variety of advantageous properties for their use in the conversion of oligosaccharides, disaccharides and/or monosaccharides of hexoses and polysaccharides, oligosaccharides, disaccharides and/or monosaccharides of pentoses, in particular derived from lignocellulosic hydrolysates to high level of lactic acid while producing low level of acetic acid.

It is an advantage of the methods according to the present disclosure that the used microorganisms are able to convert highly complex polysaccharides like xylan to high yields of carbon-based chemicals like lactate.

In particular, these used microorganisms are extreme thermophiles and show a broad substrate specificities and high natural production of lactic acid. Therefore, this allows an lactate fermentation at very high temperatures, for example over 70° C., which has many advantages over mesophilic fermentation. One advantage of thermophilic fermentation is the minimization of the problem of contamination in continuous cultures, since only a few microorganisms are able to grow at such high temperatures in un-detoxified lignocellulose hydrolysate.

In the present context the term “lignocellulosic hydrolysate” is intended to designate a lignocellulosic biomass that has been subjected to a pre-treatment step whereby lignocellulosic material has been at least partially separated into cellulose, hemicellulose and lignin thereby having increased the surface area of the material. The lignocellulosic material may typically be derived from plant material, such as straw, hay, garden refuse, comminuted wood, fruit hulls and seed hulls.

The term “a microorganism” as used herein may refer to only one unicellular organism as well as to numerous single unicellular organisms. For example, the term “a microorganism of the genus Thermoanaerobacter” may refer to one single Thermoanaerobacter bacterial cell of the genus Thermoanaerobacter as well as to multiple bacterial cells of the genus Thermoanaerobacter.

The terms “a strain of the genus Thermoanaerobacter” and “a Thermoanaerobacter cell” are used synonymously herein. In general, the term “microorganisms” refers to numerous cells.

In particular, said term refers to at least 10³ cells, preferably at least 10⁴ cells, at least 10⁵ or at least 10⁶ cells.

A strain “homolog” as used herein is considered any bacterial strain, which is not significantly different by means of DNA homology as defined above and exhibits same or comparable physiological properties as described in the examples herein.

The term “mutant” as used herein refers to a bacterial cell in which the genome, including one or more chromosomes or potential extra-chromosomal DNA, has been altered at one or more positions, or in which DNA has been added or removed.

As used herein “mutant” or “homolog” means also a microorganism derived from the cells or strains according to the present disclosure, which are altered due to a mutation. A mutation is a change produced in cellular DNA, which can be spontaneous, caused by an environmental factor or errors in DNA replication, or induced by physical or chemical conditions. The processes of mutation included in this and indented subclasses are processes directed to production of essentially random changes to the DNA of the microorganism including incorporation of exogenous DNA. All mutants of the microorganisms comprise the advantages of being extereme thermophile (growing and fermenting at temperatures above 70° C.) and are capable of fermenting lignocellulosic biomass to lactic acid, salts and/or esters thereof. In an advantageous embodiment, mutants of the microorganisms according to the present disclosure have in a DNA-DNA hybridization assay, a DNA-DNA relatedness of at least 80%, preferably at least 90%, at least 95%, more preferred at least 98%, most preferred at least 99%, and most preferred at least 99.9% with one of the isolated bacterial strains DIB004G, DIB087G, DIB097X, DIB101G, DIB101X, DIB103X, DIB104X and DIB107X.

The term “progeny” is refers to a product of bacterial reproduction, a new organism produced by one or more parents.

As mentioned above lignocellolytic biomass according to the present disclosure can be but is not limited to grass, switch grass, cord grass, rye grass, reed canary grass, mixed prairie grass, miscanthus, Napier grass, sugar-methoding residues, sugarcane bagasse, sugarcane straw, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, pressmud from sugar beet, cotton stalk, banana leaves, oil palm residues and lignocellulosic biomass material obtained through processing of food, plants. In advantageous embodiments, the lignocellulosic biomass material is hardwood and/or softwood, preferably poplar wood. In advantageous embodiments, the lignocellulosic biomass material is a grass or perennial grass, preferably miscanthus.

The term “DNA-DNA relatedness” in particularly refers to the percentage similarity of the genomic or entire DNA of two microorganisms as measured by the DNA-DNA hybridization/renaturation assay according to De Ley et al. (1970) Eur. J. Biochem. 12, 133-142 or Huβ et al. (1983) Syst. Appl. Microbiol. 4, 184-192. In particular; the DNA-DNA hybridization assay preferably is performed by the DSMZ (Deutsche Sammlung von Mikroorganismen and Zellkulturen GmbH, Braunschweig, Germany) Identification Service.

The term “16S rDNA gene sequence similarity” in particular refers to the percentage of identical nucleotides between a region of the nucleic acid sequence of the 16S ribosomal RNA (rDNA) gene of a first microorganism and the corresponding region of the nucleic acid sequence of the 16S rDNA gene of a second microorganism. Preferably, the region comprises at least 100 consecutive nucleotides, more preferably at least 200 consecutive nucleotides, at least 300 consecutive nucleotides or at least 400 consecutive nucleotides, most preferably about 480 consecutive nucleotides.

The strains according to disclosure have the potential to be capable of producing high levels of carboxylic acids like lactic acid, propionic acid, acetic acid, succinic acid, butyric acid or formic acid.

In advantageous embodiments, the lignocellulosic biomass material is subjected to mechanical, thermochemical, and/or biochemical pretreatment. The lignocellulosic biomass material could be exposed to steam treatment. In further embodiments, the lignocellulosic biomass material is pretreated with mechanical comminution and a subsequent treatment with lactic acid, acetic acid, sulfuric acid or sulfurous acid or their respective salts or anhydrides under heat and pressure with or without a sudden release of pressure. In another embodiment, the lignocellulosic biomass material is pretreated with mechanical comminution and a subsequent treatment with either sodium hydroxide, ammonium hydroxide, calcium hydroxide or potassium hydroxide under heat and pressure with or without a sudden release of pressure.

In advantageous embodiments, the lignocellulosic biomass material is pretreated with mechanical comminution and subsequent exposure to a multi-step combined pretreatment process. Such multi-step combined pretreatment may include a treatment step consisting of cooking in water or steaming of the lignocellulosic biomass material at a temperature of 100-200° C. for a period of time in between 5 and 120 min. Suitable catalysts including but not limited to lactic acid, acetic acid, sulfuric acid, sulfurous acid, sodium hydroxide, ammonium hydroxide, calcium hydroxide or potassium hydroxide or their respective salts or anhydrides may or may not be added to the process. The process may further include a step comprising a liquid-solid separation operation, e.g. filtration, separation, centrifugation or a combination thereof, separating the process fluid containing partially or fully hydrolyzed and solubilized constituents of the lignocellulosic biomass material from the remaining insoluble parts of the lignocellulosic biomass. The process may further include a step comprising washing of the remaining lignocellulosic biomass material. The solid material separated from solubilized biomass constituents may then be treated in a second step with steam under heat and pressure with or without a sudden release of pressure at a temperature of 150-250° C. for a period of time in between 1 and 15 min. In order to increase pretreatement effectiveness, a suitable catalyst including but not limited to lactic acid, acetic acid, sulfuric acid, sulfurous acid, sodium hydroxide, ammonium hydroxide, calcium hydroxide or potassium hydroxide or their respective salts or anhydrides may be added also to the second step.

In advantageous embodiments, the lignocellulosic biomass is milled before converted into carboxylic acids like lactic acid. In one embodiment, the lignocellulosic biomass is pretreated biomass from Populus sp, preferably pretreated with steam pretreatment or multi-step combined pretreatment. In another embodiment, the lignocellulosic biomass is pretreated biomass from any perennial grass, e.g. Miscanthus sp., preferably treated with steam pretreatment or multi-step combined pretreatment.

In further advantageous embodiments the lignocellulosic hydrolysate is then treated with an enzymatic hydrolysis with one or more appropriate carbohydrase enzymes such as cellulases, glucosidases and/or hemicellulases including xylanases.

The pretreatment method most often used is steam pretreatment, a process comprising heating of the lignocellulosic material by steam injection to a temperature of 130-230 degrees centigrade with or without subsequent sudden release of pressure. Prior to or during steam pretreatment, a catalyst like a mineral or organic acid or a caustic agent facilitating disintegration of the biomass structure can be added optionally. Catalysts often used for such a pretreatment include but are not limited to sulphuric acid, sulphurous acid, hydrochloric acid, acetic acid, lactic acid, sodium hydroxide (caustic soda), potassium hydroxide, calcium hydroxide (lime), ammonia or the respective salts or anhydrides of any of these agents.

Such steam pretreatment step may or may not be preceded by another treatment step including cooking of the biomass in water or steaming of the biomass at temperatures of 100-200° C. with or without the addition of a suitable catalyst like a mineral or organic acid or a caustic agent facilitating disintegration of the biomass structure. In between the cooking step and the subsequent steam pretreatment step one or more liquid-solid-separation and washing steps can be introduced to remove solubilized biomass components in order to reduce or prevent formation of inhibitors during the subsequent steam pretreatment step. Inhibitors formed during heat or steam pretreatment include but are not limited to furfural formed from monomeric pentose sugars, hydroxymethylfurfural formed from monomeric hexose sugars, acetic acid, levulinic acid, phenols and phenol derivatives.

Another type of lignocellulose hydrolysis is acid hydrolysis, where the lignocellulosic material is subjected to an acid such as sulfuric acid or sulfurous acid whereby the sugar polymers cellulose and hemicellulose are partly or completely hydrolysed to their constituent sugar monomers. A third method is wet oxidation wherein the material is treated with oxygen at 150-185 degrees centigrade. The pretreatments can be followed by enzymatic hydrolysis to complete the release of sugar monomers. This pre-treatment step results in the hydrolysis of cellulose into glucose while hemicellulose is transformed into the pentoses xylose and arabinose and the hexoses glucose, mannose and galactose. The pretreatment step may in certain embodiments be supplemented with treatment resulting in further hydrolysis of the cellulose and hemicellulose. The purpose of such an additional hydrolysis treatment is to hydrolyze oligosaccharide and possibly polysaccharide species produced during the acid hydrolysis, wet oxidation, or steam pretreatment of cellulose and/or hemicellulose origin to form fermentable sugars (e.g. glucose, xylose and possibly other monosaccharides). Such further treatments may be either chemical or enzymatic. Chemical hydrolysis is typically achieved by treatment with an acid, such as treatment with aqueous sulphuric acid or hydrochloric acid, at a temperature in the range of about 100-150 degrees centigrade. Enzymatic hydrolysis is typically performed by treatment with one or more appropriate carbohydrase enzymes such as cellulases, glucosidases and hemicellulases including xylanases.

It has been found that the microorganisms according to the present disclosure can grow efficiently on various types of pretreated and untreated biomass (e.g. wood incl. poplar, spruce and cotton wood; various types of grasses and grass residues incl. miscanthus, wheat straw, sugarcane bagasse, corn stalks, corn cobs, whole corn plants, sweet sorghum). As used herein “efficient” growth refers to growth in which cells may be cultivated to a specified density within a specified time.

The microorganisms according to the present disclosure can grow efficiently on hydrolysis products of cellulose (e.g. disaccharide cellobiose), cellulose derived hexoses (e.g. glucose), unhydrolyzed, hemicelluloses like xylan, hemicellulose derived pentoses (e.g. xylose), unhydrolyzed amyloseas well as steam pretreated poplar or miscanthus. In particular, one of the main products when grown on cellobiose, glucose and xylose is lactic acid. One of the main products when growing on pretreated biomass substrates is also lactic acid or a salt or ester thereof (like lactate) for example, when the microorganisms were grown on steam-pretreated poplar wood or miscanthus grass the lactate yield is high. The microorganisms according to the present disclosure also grew efficiently on cellobiose.

Cellobiose is a disaccharide derived from the condensation of two glucose molecules linked in a β(1→4) bond. It can be hydrolyzed to give glucose. Cellobiose has eight free alcohol (OH) groups, one either linkage or two hemiacetal linkages, which give rise to strong inter- and intra-molecular hydrogen bonds. It is a type of dietary carbohydrate also found in mushrooms.

Xylan is a generic term used to describe a wide variety of highly complex polysaccharides that are found in plant cell walls and some algae. Xylans are polysaccharides made from units of xylose.

Furthermore, the microorganisms according to the present disclosure grew efficiently on the soluble materials obtained after heat-treating of lignocellulosic biomass.

The microorganisms according to the invention are anaerobic thermophile bacteria, and they are capable of growing at high temperatures even at or above 70 degrees centigrade The fact that the strains are capable of operating at this high temperature is of high importance in the conversion of the lignocellulosic hydrolysates into fermentation products. The conversion rate of carbohydrates into e.g. lactic acid is much faster when conducted at high temperatures. A smaller production plant is required for a given plant capacity, thereby reducing plant construction costs. As also mentioned previously, the high temperature reduces the risk of contamination from other microorganisms, resulting in less downtime, increased plant productivity and a lower energy requirement for feedstock sterilization. The high operation temperature may also facilitate the subsequent recovery of the resulting fermentation products.

Lignocellulosic biomass material and lignocellulose hydrolysates contain inhibitors such as furfural, phenols and carboxylic acids, which can potentially inhibit the fermenting organism. Therefore, it is an advantage of the microorganisms according to the present disclosure that they are tolerant to these inhibitors.

The microorganisms according to the present disclosure are novel species of the genus Thermoanaerobacter or novel subspecies of Thermoanaerobacter mathranii.

For example, the genus Thermoanaerobacter includes different species of extremely thermophilic (temperature optima for growth higher than 70° C.) hemicellulolytic and saccharolytic strictly anaerobic bacteria (Lee et al. 1993). Thermoanaerobacter mathranii DSM 11426 is an extremely thermophilic bacterium. It has a temperature optimum between 70 and 75° C. and was isolated from a hot spring in Iceland (Larsen et al. 1997). It uses a number of sugars including xylan as carbon sources, but did not utilize microcrystalline cellulose. Fermentation end products on xylose were ethanol, acetate, low amounts of lactate, CO₂, and H₂ (Larsen et al. 1997).

According to the present disclosure, the microorganisms are used to produce lactic acid, a salt or an ester thereof, wherein the methods show several features that distinguish them from currently known methods: (i) high yield and low product inhibition, (ii) simultaneous utilization of lignocellolosic biomass material derived sugars, and (iii) microorganism growth at elevated temperatures.

The used microorganisms in the methods according to the present disclosure are robust thermophilic organisms with a decreased risk of contamination. They efficiently convert an extraordinarily wide range of biomass components to carbon-based chemicals like lactic acid.

As mentioned above, in one aspect, the present disclosure relates to the use of an isolated cell and/or strain comprising a 16S rDNA sequence selected from the group consisting of SEQ ID NO 1, SEQ ID NO 2, SEQ ID NO 3, SEQ ID NO 4, SEQ ID NO 5, SEQ ID NO 6, SEQ ID NO 7 and SEQ ID NO 8, or any combination thereof.

In one aspect, the present disclosure pertains to an isolated Thermoanaerobacter sp. cell having a 16S rDNA sequence at least 99, at least 99.3, at least 99.5, at least, 99.7, at least 99.9, at least 99.99 percent identical to SEQ ID NO 1, SEQ ID NO 2, SEQ ID NO 3, SEQ ID NO 4, SEQ ID NO 5, SEQ ID NO 6, SEQ ID NO 7 and/or SEQ ID NO 8.

In one embodiment of the present disclosure the used isolated strain is Thermoanaerobacter sp. DIB004G (DSMZ Accession number 25179), cells derived there from, mutants there from, a progeny or a Thermoanaerobacter sp. DIB004G homolog.

In another embodiment of the present disclosure the used isolated strain is Thermoanaerobacter sp. DIB087G (DSMZ Accession number 25777), cells derived there from, mutants there from, a progeny or a Thermoanaerobacter sp. DIB087G homolog.

In another embodiment of the present disclosure the used isolated strain is Thermoanaerobacter sp. DIB097X (DSMZ Accession number 25308), cells derived there from, mutants there from, a progeny or a Thermoanaerobacter sp. DIB097X homolog.

In another embodiment of the present disclosure the used isolated strain is Thermoanaerobacter sp. DIB101G (DSMZ Accession number 25180), cells derived there from, mutants there from, a progeny or a Thermoanaerobacter sp. DIB101G homolog.

In another embodiment of the present disclosure the used isolated strain is Thermoanaerobacter sp. DIB101X (DSMZ Accession number 25181), cells derived there from, mutants there from, a progeny or a Thermoanaerobacter sp. DIB101X homolog.

In another embodiment the used isolated strain is Thermoanaerobacter sp. DIB103X (DSMZ Accession number 25776), cells derived there from, mutants there from, a progeny or a Thermoanaerobacter sp. DIB103X homolog.

In another embodiment the used isolated strain is Thermoanaerobacter sp. DIB104X (DSMZ Accession number 25778), cells derived there from, mutants there from, a progeny or a Thermoanaerobacter sp. DIB104X homolog.

In another embodiment the used isolated strain is Thermoanaerobacter sp. DIB107X (DSMZ Accession number 25779), cells derived there from, mutants there from, a progeny or a Thermoanaerobacter sp. DIB107X homolog.

The invention is based on the isolated bacterial strains Thermoanaerobacter sp. as listed in table 1 that contain 16S rDNA sequences 100 percent and/or 99.99 percent identical to the respectively list sequences.

TABLE 1

| | | | DSMZ | | 16SrDNA |
|---|---|---|---|---|---|
| | | | accession | Deposition | SEQ ID |
| Genus | Species | Name | number | date | NO. |
| Thermoanaerobacter | sp. | DIB004G | DSM 25179 | 15 Sep. 2011 | 1 |
| Thermoanaerobacter | sp. | DIB087G | DSM 25777 | 15 Mar. 2012 | 2 |
| Thermoanaerobacter | sp. | DIB097X | DSM 25308 | 27 Oct. 2011 | 3 |
| Thermoanaerobacter | sp. | DIB101G | DIB 25180 | 15 Sep. 2011 | 4 |
| Thermoanaerobacter | sp. | DIB101X | DSM 25181 | 15 Sep. 2011 | 5 |
| Thermoanaerobacter | sp. | DIB103X | DSM 25776 | 15 Mar. 2012 | 6 |
| Thermoanaerobacter | sp. | DIB104X | DSM 25778 | 15 Mar. 2012 | 7 |
| Thermoanaerobacter | sp. | DIB107X | DSM 25779 | 15 Mar. 2012 | 8 |

All strains as listed in table 1 have been deposited in accordance with the terms of the Budapest Treaty on Sep. 15, 2011 with DSMZ—Deutsche Sammlung von Mikroorganismen and Zellkulturen GmbH, Inhoffenstr. 7B, 38124 Braunschweig, Germany—under the respectively indicated accession numbers and deposition dates by DIREVO Industrial Biotechnology GmbH, Nattermannallee 1, 50829 Cologne, Germany (DE).

As is apparent from the following, the preferred strains of the present disclosure have been deposited. Other cells, strains, bacteria, microorganisms and/or microbial cultures of the present disclosure can therefore be obtained by mutating the deposited strains and selecting derived mutants having enhanced characteristics. Desirable characteristics include an increased range of sugars that can be utilized, increased growth rate, ability to produce higher amounts of fermentation products such as lactic acid, etc. Suitable methods for mutating bacteria strains and selecting desired mutants are described in Functional analysis of Bacterial genes: A practical Manual, edited by W. Schumann, S. D. Ehrlich & N. Ogasawara, 2001.

The microorganisms of the species Thermoanaerobacter sp. according to the present disclosure in particular refer to a microorganism, which belongs to the genus Thermoanaerobacter and which preferably has one or more of the following characteristics:
a) it is a microorganism of the genus Thermoanaerobacter; and/or
b) in a DNA-DNA hybridization assay, it shows a DNA-DNA relatedness of at least 70%, preferably at least 90%, at least 95%, more preferred at least 98%, most preferred at least 99% with the Thermoanaerobacter sp. strains listed in table 1 with their respective accession numbers; and/or
c) it displays a level of 16S rDNA gene sequence similarity of at least 98%, preferably at least 99% or at least 99.5%, more preferably 100% with either of the Thermoanaerobacter sp. strains listed in table 1 with their respective accession numbers; and/or
d) it is capable of surviving in high temperature conditions above 70° C., and/or
e) it is a Gram-positive bacterium.

Preferably, at least two or at least three, and more preferred all of the above defined criteria a) to e) are fulfilled.

In an advantageous embodiment, the microorganisms according to the present disclosure in particular refer to a microorganism which belongs to the genus Thermoanaerobacter and which preferably has one or more of the following characteristics:
a) It is a microorganism of the genus Thermoanaerobacter
b) it is a microorganism of the species Thermoanaerobacter thermohydrosulfuricus, Thermoanaerobacter thermocopriae or Thermoanaerobacter mathranii,
c) in a DNA-DNA hybridization assay, it shows a DNA-DNA relatedness of at least 80%, preferably at least 90%, at least 95%, more preferred at least 98%, most preferred at least 99%, and most preferred at least 99.9% with one of the strains of table 1; and/or
d) it displays a level of 16S rDNA gene sequence similarity of at least 98%, preferably at least 99%, at least 99.5% or at least 99.7%, more preferably 99.99% with one of the strains listed in table 1; and/or
e) it is capable of surviving and/or growing and/or producing carboxylic acids, preferably lactic acid, salts or esters thereof at temperature conditions above 70° C., in particular of above 72° C.

Preferably, at least two or at least three, and more preferred all of the above defined criteria a) to e) are fulfilled.

The used Thermoanaerobacter sp. strains listed in table 1 in a method according to the present disclosure have several highly advantageous characteristics needed for the conversion of lignocellulosic biomass material. Thus, these base strains possess all the genetic machinery for the conversion of both pentose and hexose, sugars to various fermentation products such as lactic acid. As will be apparent from the below examples, the examination of the complete 16S rDNA sequence showed that the related strains Thermoanaerobacter sp. DIB087G, Thermoanaerobacter sp. DIB101G and Thermoanaerobacter sp. DIB104X may be related to Thermoanaerobacter thermohydrosulfuricus, although the 16S rDNA sequences clearly place them in separate subspecies or even different species. The strain Thermoanaerobacter sp. DIB107X may be related to Thermoanaerobacter thermocopriae, although the 16S rDNA sequences clearly place them in separate subspecies or even different species. The strains Thermoanaerobacter sp. DIB004G, Thermoanaerobacter sp. DIB097X, Thermoanaerobacter sp. DIB101X and Thermoanaerobacter sp. DIB103X may be related to Thermoanaerobacter mathranii, although the 16S rDNA sequences clearly place them in separate subspecies or even different species.

It is a great advantage to use the Thermoanaerobacter sp. strains listed in table 1 in a method according to the present disclosure since they are xylanolytic and saccharolytic (ferment hemicelluloses, e.g. xylan, hexoses and pentoses to lactic acid and small amounts of acetate).

In a preferred embodiment, the used Thermoanaerobacter sp. microorganism is
a) Either Thermoanaerobacter sp. listed in table 1, deposited under their respectively indicated accession number and deposition date, according to the requirements of the Budapest Treaty at the Deutsche Sammlung von Mikroorganismen and Zellkulturen (DSMZ), Inhoffenstraβe 7B, 38124 Braunschweig (DE) by DIREVO Industrial Biotechnology GmbH, Nattermannallee 1, 50829 Cologne, Germany (DE), or
b) a microorganism derived from either of these Thermoanaerobacter sp. strains, or
c) a homolog or mutant of either respective Thermoanaerobacter sp. strain

All strains Thermoanaerobacter sp. as listed in table 1 belong to the genus Thermoanaerobacter and are extremely thermophilic (growth at temperatures higher than 70° C.), xylanolytic, amylolytic and saccharolytic, strictly anaerobic, Gram-positive bacteria. Cells are straight rods 0.3-0.4 μm by 2.0-6.0 μm, occuring both singly and in pairs. These strains grow on various sugars as substrate, including starch, xylan, xylose, cellobiose, and glucose. One of the main fermentation products on these substrates is lactic acid, a salt or an ester thereof. Low amounts of acetate are also formed.

In advantageous embodiments the cells, strains, microorganisms may be modified in order to obtain mutants or derivatives with improved characteristics. Thus, in one embodiment there is provided a bacterial strain according to the disclosure, wherein one or more genes have been inserted, deleted or substantially inactivated. The variant or mutant is typically capable of growing in a medium comprising a lignocellulosic biomass material and/or a lignocellulosic hydrolysate.

In another embodiment, there is provided a process for preparing variants or mutants of the microorganisms according to the present disclosure, wherein one or more genes are inserted, deleted or substantially inactivated as described herein.

In some embodiments one or more additional genes are inserting into the strains according to the present disclosure. Thus, in order to improve the yield of the specific fermentation product, it may be beneficial to insert one or more genes encoding a polysaccharase into the strain according to the invention. Hence, in specific embodiments there is provided a strain and a process according to the invention wherein one or more genes encoding a polysaccharase which is selected from cellulases (such as EC 3.2.1.4); beta-glucanases, including glucan-1,3 beta-glucosidases (exo-1,3 beta-glucanases, such as EC 3.2.1.58), 1,4-beta-cellobiohydrolases (such as EC 3.2.1.91) and endo-I,3(4)-beta-glucanases (such as EC 3.2.1.6); xylanases, including endo-I,4-beta-xylanases (such as EC 3.2.1.8) and xylan 1,4-beta-xylosidases (such as EC 3.2.1.37); pectinases (such as EC 3.2.1.15); alpha-glucuronidases, alpha-L-arabinofuranosidases (such as EC 3.2.1.55), acetylesterases (such as EC 3.1.1.-), acetylxylanesterases (such as EC 3.1.1.72), alpha-amylases (such as EC 3.2.1.1), beta-amylases (such as EC 3.2.1.2), glucoamylases (such as EC 3.2.1.3), pullulanases (such as EC 3.2.1.41), beta-glucanases (such as EC 3.2.1.73), hemicellulases, arabinosidases, mannanases including mannan endo-1,4-beta-mannosidases (such as EC 3.2.1.78) and mannan endo-I,6-alpha-mannosidases (such as EC 3.2.1.101), pectin hydrolases, polygalacturonases (such as EC 3.2.1.15), exopolygalacturonases (such as EC 3.2.1.67) and pectate lyases (such as EC 4.2.2.10), are inserted.

In accordance with the present disclosure, a method of producing a fermentation product comprising culturing a strain according to the invention under suitable conditions is also provided.

The strains according to the disclosure are strictly anaerobic microorganisms, and hence it is preferred that the fermentation product is produced by a fermentation process performed under strictly anaerobic conditions. Additionally, the strain according to invention is an extremely thermophillic microorganism, and therefore the process may perform optimally, when it is operated at temperature in the range of about 40-95 degrees centigrade, such as the range of about 50-90 degrees centigrade, including the range of about 60-85 degrees centigrade, such as the range of about 65-75 degrees centigrade

For the production of certain fermentation products, it may be useful to select a specific fermentation process, such as batch fermentation process, including a fed-batch process or a continuous fermentation process. Also, it may be useful to select a fermentation reactor such as an immobilized cell reactor, a fluidized bed reactor or a membrane bioreactor.

In an advantageous embodiment, the conversion of hydrolyzed lignocellulosic biomass to lactic acid is realized in a single step process as part of a consolidated bioprocessing (CBP) system.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements does not comprise further elements as well as embodiments wherein the subject-matter which “comprises” specifically listed elements may and/or indeed does encompass further elements. Likewise, the expression “have” is to be understood as the expression “comprise”, also including and specifically referring to the expressions “consist essentially of” and “consist of”.

The following methods and examples are offered for illustrative purposes only, and are not intended to limit the scope of the present disclosure in any way.

### METHODS AND EXAMPLES

In the following examples, materials and methods of the present disclosure are provided including the determination of properties of the strains according to the present disclosure. It should be understood that these examples are for illustrative purpose only and are not to be construed as limiting this disclosure in any manner. All publications, patents, and patent applications cited herein are hereby incorporated by reference in their entirety for all purposes.

### Example 1

### Isolation and Cultivation

All procedures for enrichment and isolation of strains listed in table 1 employed anaerobic technique for strictly anaerobic bacteria (Hungate 1969). The strains were enriched from environmental samples at temperatures higher than 70° C. with crystalline cellulose and beech wood as substrate. Isolation was performed by serial dilutions in liquid media with xylan as substrate followed by picking colonies grown on solid agar medium at 72° C. in Hungate roll tubes (Hungate 1969).

The cells are cultured under strictly anaerobic conditions applying the following medium:

Basic medium

| | | |
|---|---|---|
| NH4Cl | 1.0 | g |
| NaCl | 0.5 | g |
| MgSO4 × 7 H2O | 0.3 | g |
| CaCl2 × 2 H2O | 0.05 | g |
| NaHCO3 | 0.5 | g |
| K2HPO4 | 1.5 | g |
| KH2PO4 | 3.0 | g |
| Yeast extract (bacto, BD) | 0.5 | g |
| Cellobiose | 5.0 | g |
| Vitamins (see below) | 1.0 | ml |
| Trace elements (see below) | 0.5 | ml |
| Resazurin | 1.0 | mg |
| Na2S × 9 H2O | 0.75 | g |
| Distilled water | 1000.0 | ml |

Trace elements stock solution

| | | |
|---|---|---|
| NiCl₂ × 6H₂O | 2 | g |
| FeSO₄ × 7H₂O | 1 | g |
| NH₄Fe(III) citrate, brown, 21.5% Fe | 10 | g |
| MnSO₄ × H₂O | 5 | g |
| CoCl₂ × 6H₂O | 1 | g |
| ZnSO₄ × 7H₂O | 1 | g |
| CuSO₄ × 5H₂O | 0.1 | g |
| H₃BO₃ | 0.1 | g |
| Na₂MoO₄ × 2H₂O | 0.1 | g |
| Na₂SeO₃ × 5H₂O | 0.2 | g |
| Na₂WoO₄ × 2H₂O | 0.1 | g |
| Distilled water | 1000.0 | ml |

Add 0.5 ml of the trace elements stock solution
to 1 liter of the medium

Vitamine stock solution

| | | |
|---|---|---|
| nicotinic acid | 200 | mg |
| cyanocobalamin | 25 | mg |
| p-aminobenzoic acid (4-aminobenzoic acid) | 25 | mg |
| calcium D-pantothenate | 25 | mg |
| thiamine-HCl | 25 | mg |
| riboflavin | 25 | mg |
| lipoic acid | 25 | mg |
| folic acid | 10 | mg |
| biotin | 10 | mg |
| pyridoxin-HCl | 10 | mg |
| Distilled water | 200.0 | ml |

Add 1 ml of the vitamine stock solution to 1 liter of the medium

All ingredients except sulfide are dissolved in deionized water and the medium is flushed with nitrogen gas (purity 99.999%) for 20 min at room temperature. After addition of sulfide, the pH-value is adjusted to 7.0 at room temperature with 1 M HCl. The medium is then dispensed into Hungate tubes or serum flasks under nitrogen atmosphere and the vessels are tightly sealed. After autoclaving at 121° C. for 20 min pH-value should be in between 6.8 and 7.0.

Soluble sugar substrates (xylose, cellobiose, glucose) as specified for individual experiments are added sterile filtered after autoclaving. Xylan is autoclaved with the medium. Subsequent to autoclaving, cultures are inoculated by injection of a seed culture through the seal septum and inoculated in an incubator at 72° C. for the time indicated.

### Example 2

### HPLC

Sugars and fermentation products were quantified by HPLC-RI using a Via Hitachi LaChrom Elite (Hitachi corp.) fitted with a Rezex ROA Organic Acid H+ (Phenomenex). The analytes were separated isocratically with 2.5 mM H₂S0₄ and at 65° C.

### Example 3

### Phylogenetic Analysis of 16S rDNA Genes

Genomic DNA was isolated from cultures grown as described above and 16SrDNA amplified by PCR using 27F (AGAGTTTGATCMTGGCTCAG; SEQ ID NO. 9) as forward and 1492R (GGTTACCTTGTTACGACTT; SEQ ID NO. 9) as reverse primer. The resulting products were sequenced and the sequences analyzed using the Sequencher 4.10.1 software (Gene Codes Corporation). The NCBI database was used for BLAST procedures.

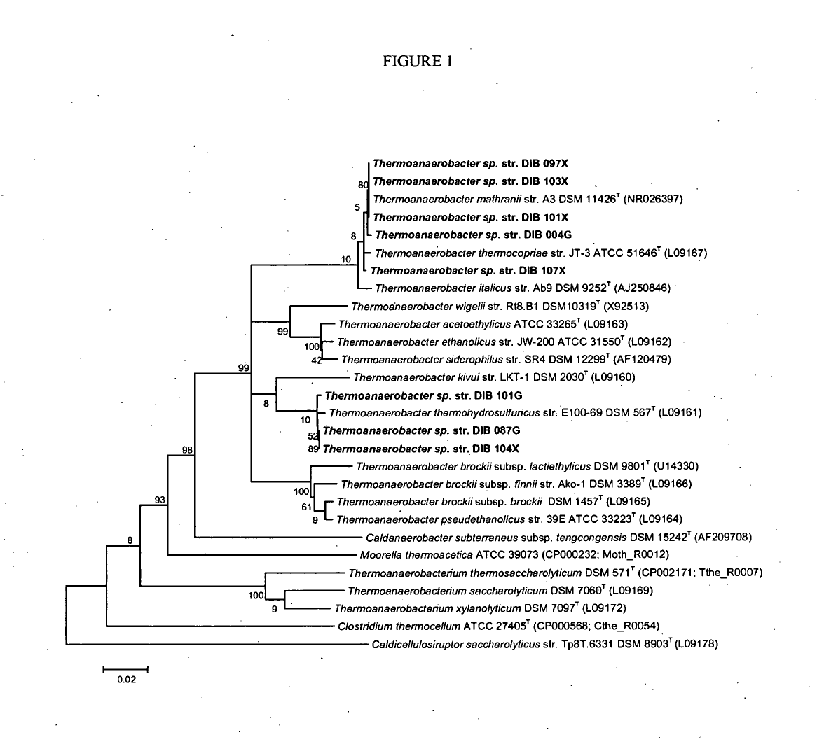

Alignment was carried out using ClustalW (Chenna et al. 2003) and the phylogenetic tree was constructed using software MEGA4 (Kumar et al. 2001). The tree for all strains listed in table 1 is displayed in FIG. 1.

### Example 4

### Production of Lactate on Different Substrates

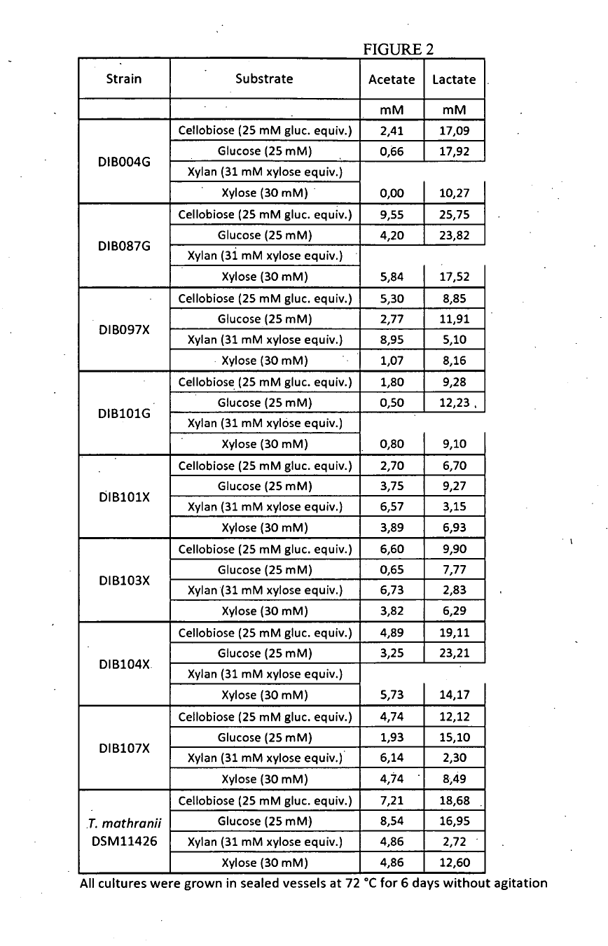

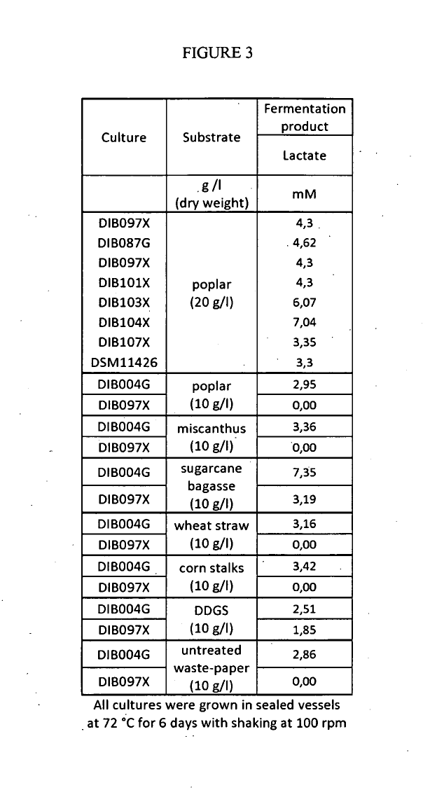

Experiments on growth and fermentation on cellobiose, glucose, xylan and xylose as well as on pretreated poplar wood, miscanthus grass, sugarcane bagasse, wheat straw, corn stalks and DDGS as well as on non-pretreated waste paper were performed by cultivation in sealed 16 ml tubes with 8 ml medium described in Example 1. All strains grew well on these substrates (FIGS. 2 and 3) except strains DIB004G, DIB087G and DIB101G which did not grow on xylane. No growth was detected on cellulose. Only small amounts of acetate were formed (FIGS. 2 and 3). In contrast, the known lactate-producing thermophilic bacterium Thermoanaerobacter mathranii strain A3 (DSM 11426) (Larsen et al 1997) produced lower amounts of lactate as well as higher amounts of acetate.

### Example 5

### Fermentation

Batch experiments with all strains; e.g. DIB004G, were performed by cultivation on the medium described above with addition of the respectively indicated substrate, e.g. 20 g/L miscanthus grass pretreated with a suitable method selected from those described above comprising heating in the presence of dilute acid followed by sudden release of pressure.

Temperature is controlled to 72° C. and the pH-value is controlled to 6.75±0.1 throughout the fermentation. The fermenter is purged with nitrogen to remove excess oxygen before sodium sulphide is added as described above.

The fermentation is started by addition of a seed culture prepared as described in example 1.

The results of the HPLC analysis as described in example 2 show parallel production of lactatic acid and acetic acid.

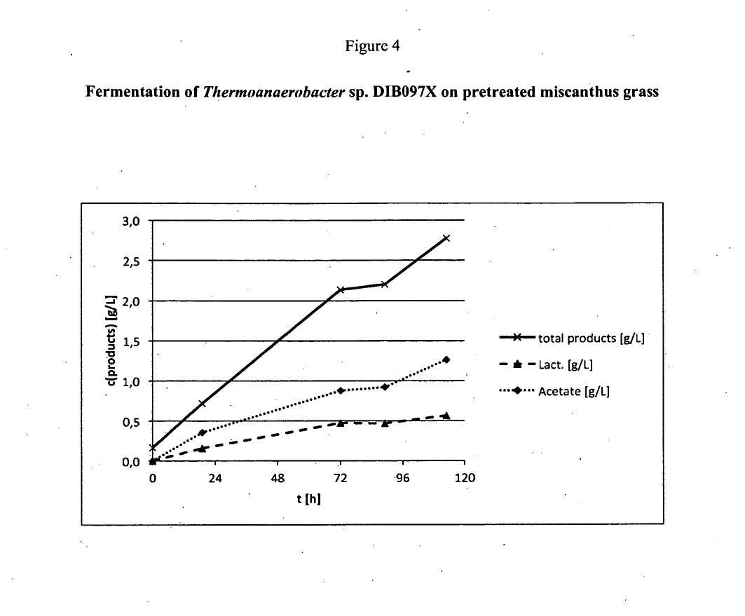

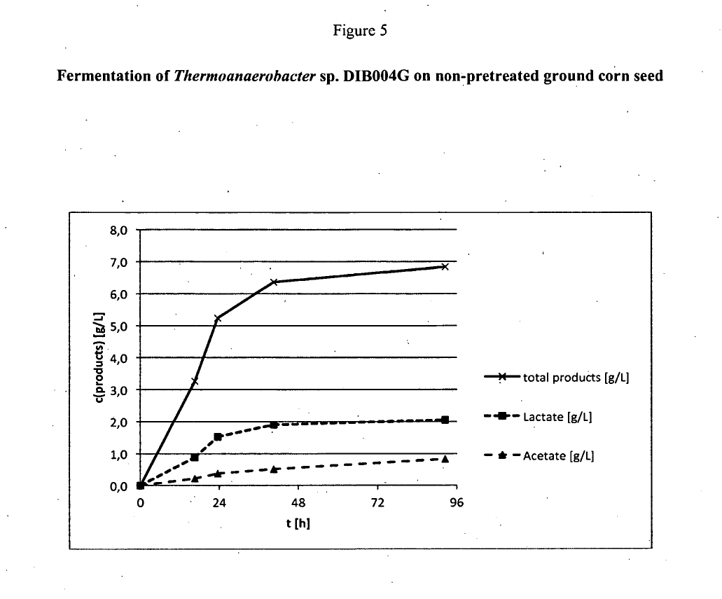

The results for product formation during a fermentation of Thermoanaerobacter sp. DIB097X on pretreated miscanthus grass is shown in FIG. 4. The results for product formation during a fermentation of Thermoanaerobacter sp. DIB004G on non-pretreated ground corn seed is shown in FIG. 5.

### LIST OF ADDITIONAL REFRENCES

Lee Y-E, Jain M K, Lee c. Lowe S E, Zeikus J G (1993) Taxonomic distinction of saccharolytic thermophilic anaerobes: Description of Thermoanaerobacterium xylanolyticum gen. nov., sp. nov., and Thermoanaerobacterium saccharolyticum gen. nov., sp.nov.; Reclassification of Thermoanaerobium brockii, Clostridium thermosulfurogenes, and Clostridium thermohydrosulfiricum EIO0-69 as Thermoanaerobacter brockii comb. nov., Thermoanaerobacterium thermosulfurigenes comb. nov., and Thermoanaerobacter thermohydrosulfuricus comb. nov., respectively; and transfer of Clostridium hermohydrosulfuricum 39E to Thermoanaerobacter ethanolicus. Int J Syst Bacteriol 43:41-51.

Larsen L, Nielsen P, Ahring B K. (1997) Thermoanaerobacter mathranii sp. nov., an ethanol-producing, extremly thermophilic anaerobic bacterium from a hot spring in Iceland. Arch Microbiol 168:114-119.
Hungate R E. (1969) A roll tube method for cultivation of strict anaerobes. In: Methods in Microbiology Eds. Norris J R and Ribbons D W. pp 118-132. New York: Academic Press.
Chenna R, Sugawara H, Koike T, Lopez R, Gibson T J, Higgins D G, Thompson J D. (2003) Multiple sequence alignment with the Clustal series of programs. Nucleic Acids Res. 13:3497-3500.
Kumar S, Tamura K, Jakobsen I B, Nei M. (2001) MEGA2: molecular evolutionary genetics analysis software. Bioinformatics. 17:1244-1245.
U.S. Pat. No. 6,555,350
International patent application WO 2007/134607

1-11. (canceled)

12. A method for converting hydrolyzed lignocellulosic biomass material to lactic acid, a salt thereof, or an ester thereof, comprising the step of contacting the hydrolyzed lignocellulosic biomass material with a microbial culture for a period of time at an initial temperature and an initial pH, thereby producing an amount of the lactic acid, wherein the microbial culture comprises an extremely thermophilic strain of the genus Thermoanaerobacter, wherein the conversion is realized in a single step process as part of a consolidated bioprocessing (CBP) system.

13. (canceled)

14. The method according to claim 12, wherein the extremely thermophilic strain is an isolated Thermoanaerobacter sp. strain comprising a 16S rDNA sequence selected from the group consisting of SEQ ID NO 1, SEQ ID NO. 2, SEQ ID NO. 3, SEQ ID NO. 4, SEQ ID NO. 5, SEQ ID NO. 6, SEQ ID NO. 7, and SEQ ID NO 8.

15. The method according to claim 12, wherein the 16S rDNA sequence is at least 99%, 99.2%, 99.4%, 99.6%, 99.8%, or at least 99.9% identical to SEQ ID NO 1, SEQ ID NO. 2, SEQ ID NO. 3, SEQ ID NO. 4, SEQ ID NO. 5, SEQ ID NO. 6, SEQ ID NO. 7, or SEQ ID NO 8.

16. The method according to claim 12, wherein the extremely thermophilic strain is DIB004G (DSMZ Accession number 25179) or DIB097X (DSMZ Accession number 25308), microorganisms derived therefrom, progenies, or mutants thereof.

17. The method according to claim 12, wherein the strain is selected from the group consisting of isolated Thermoanaerobacter sp. DIB087G, deposited as DSM 25777, Thermoanaerobacter sp. DIB101G, deposited as DSM 25180, Thermoanaerobacter sp. DIB101X, deposited as DSM 25181, Thermoanaerobacter sp DIB103X, deposited as DSM 25776, Thermoanaerobacter sp. DIB DIB104X, deposited as DSM 25778, or Thermoanaerobacter sp. DIB107X, deposited as DSM 25779, microorganisms derived therefrom, progenies, or mutants thereof.

18. The method according to claim 12, wherein the period of time is 10 h to 300 h, 50 h to 200 h, or 80 h to 160 h.

19. The method according to claim 12, wherein the initial temperature is between 55° C. and 80° C., or between 72° C. and 78° C.

20. The method according to claim 12, wherein the initial pH is between 5 and 9, or between 6 and 8.

21. The method according to claim 12, wherein the hydrolyzed lignocellulosic biomass material is derived from lignocellulosic biomass selected from the group consisting of grass, switch grass, cord grass, rye grass, reed canary grass, mixed prairie grass, miscanthus, sugar-methoding residues, sugarcane bagasse, sugarcane straw, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, and softwood, pressmud from sugar beet, cotton stalk, banana leaves, and lignocellulosic biomass material obtained through processing of food plants.

22. The method according to claim 12, wherein the hydrolyzed lignocellulosic biomass material is derived from miscanthus.

23. (canceled)

24. The method according to claim 12, wherein the hydrolyzed lignocellulosic biomass material is derived from lignocellulosic biomass selected from the group consisting of corn stover, sugarcane bagasse, cotton stalks, switchgrass, and poplar wood.

25-30. (canceled)

31. The method according to claim 12, wherein the hydrolyzed lignocellulosic biomass material is derived from lignocellulosic biomass by mechanical, thermochemical, and/or biochemical pretreatment.

32. The method according to claim 12, wherein pretreating the lignocellulosic biomass material comprises exposing the lignocellulosic biomass to steam treatment.

33. The method according to claim 12, wherein pretreating the lignocellulosic biomass material comprises exposing the lignocellulosic biomass to steam treatment and enzymatic treatment, preferably with cellulose and/or hemicellulose degrading enzymes.

34. The method according to claim 12, wherein pretreating the lignocellulosic biomass material comprises mechanical comminution and a subsequent treatment with sulfurous acid or its anhydride under heat and pressure with a sudden release of pressure.

35. The method according to claim 12, wherein pretreating the lignocellulosic biomass material comprises mechanical commination and a subsequent treatment with sulfurous acid or its anhydride under heat and pressure with a sudden release of pressure.

36. The method according to claim 12, wherein the hydrolyzed lignocellulosic biomass material is pretreated lignocellulosic biomass from Populus sp, preferably pretreated with steam explosion and enzymes, preferably with cellulose and/or hemicellulose degrading enzymes.

37. The method according to claim 12, wherein pretreating the lignocellulosic biomass comprises milling the lignocellulosic biomass.

38. (canceled)
